(19) United States
(12) Patent Application Publication
(10) Pub. No.: US 2022/0381754 A1
(43) Pub. Date: Dec. 1, 2022

(54) MICROFLUID ANALYSIS METHOD AND DEVICE FOR QUANTIFYING SOLUBLE GASEOUS POLLUTANTS IN WATER

(71) Applicants: CENTRE NATIONAL DE LA RECHERCHE SCIENTIFIQUE, PARIS (FR); IN' AIR SOLUTIONS, STRASBOURG (FR); UNIVERSITE DE STRASBOURG, STRASBOURG CEDEX (FR); ASSOCIATION POUR LA RECHERCHE ET LE DEVELOPPEMENT DE METHODES ET PROCESSUS INDUSTRIELS- ARMINES, PARIS Cedex 06 (FR)

(72) Inventors: Stéphane LE CALVE, STRASBOURG (FR); Christina ANDRIKOPOULOU, STRASBOURG (FR); Anais BECKER, STRASBOURG (FR); Pierre BERNHARDT, STRASBOURG (FR); Claire TROCQUET, STRASBOURG (FR); Hervé PLAISANCE, PARIS (FR)

(21) Appl. No.: 17/619,581

(22) PCT Filed: Jun. 22, 2020
(86) PCT No.: PCT/EP2020/067360

§ 371 Date: Dec. 19, 2021

(30) Foreign Application Priority Data
Jun. 25, 2019 (FR) FR1906855

(51) Int. Cl. G01N 33/00; G01N 21/03; G01N 21/64; G01N 21/25

(52) U.S. Cl. CPC G01N 33/0013; G01N 33/0047; G01N 21/0332; G01N 21/64; G01N 21/25

(57) ABSTRACT

A method for analyzing a gaseous pollutant by means of a microfluid circuit includes a means for pumping a liquid and a means for trapping a gas, comprising the following steps: a) generating a flow of a liquid, the liquid comprising a selective derivative agent; b) trapping and dissolving gaseous pollutant in the flow; c) reaction of the pollutant with the selective derivative agent so as to form a liquid derivative compound; d) measuring the concentration of liquid derivative compound and determining the concentration of gaseous pollutant.

The present invention relates to a method for analyzing a gaseous pollutant, such as formaldehyde, and to a device for implementing this method.

Gaseous pollutants, such as formaldehyde, are present in our environment. In the outdoor environment, they may come directly from industrial or automotive emissions or from forest fires, or indirectly by oxidation of volatile organic compounds. Formaldehyde is soluble in water and hence is also found in the oceans, seas, surface water bodies or rain water.

It is likewise found in the indoor environment, as it is given off by certain paints, by treated woods or papers, by resins or even by fabrics. In general, in an indoor environment, formaldehyde is present at concentrations of between 10 and 100 μg/m³, which may even reach several hundred μg/m³ in a working situation. Standards have set limiting thresholds on formaldehyde concentration for working and nonworking situations, and consequently the ability for precise measurement of the levels of emission of gaseous pollutants, especially of formaldehyde, in the air is becoming necessary and essential.

A number of analytical devices have already been developed. In some cases, such as the analyzers sold by Aerolaser (Hak et al., Atmos. Chem. Phys., 5: 2881-2900, 2005) or In'Air Solutions (Guglielmini et al., Talanta, 72: 102-108, 2017), they involve trapping the gaseous pollutant for analysis in a liquid solution comprising a selective derivatizing agent with which the gaseous pollutant gives a quantitative reaction. The concentration of gaseous pollutant can be measured via the measurement of the concentration of the product resulting from the reaction between the derivatizing agent and the gaseous pollutant. Nevertheless, these devices comprise a gas pump, for trapping the air comprising the gaseous pollutant for analysis in the device, and a mass flow regulator, for regulating the flow rate of the liquid solution comprising the selective derivatizing agent, which are particularly expensive, bulky, noisy and energy-consuming. This is limiting the development of portable analytical devices.

The invention aims to remedy the above-stated drawbacks in the prior art, being aimed more particularly at providing a method for analyzing a gaseous pollutant and a device for analyzing a gaseous pollutant that is able to implement the method, the device comprising neither any gas pump nor any mass flow regulator.

The invention therefore provides a method for analyzing a gaseous pollutant by means of a microfluidic circuit comprising a means for pumping a liquid and a means for trapping a gas, characterized in that it comprises the following steps:
a) generating a flow of a liquid, the liquid comprising a selective derivatizing agent;
b) trapping and dissolving the gaseous pollutant in the flow;
c) reacting the pollutant with the selective derivatizing agent to form a liquid derivative compound;
d) measuring the concentration of liquid derivative compound and determining the concentration of gaseous pollutant.

According to embodiments of the invention:

step c) comprises temperature-regulating the liquid flow;

step d) is carried out by fluorescence spectroscopy or by colorimetry;

the gaseous pollutant is selected from a compound from the class of the aldehydes or a compound from the class of the chloramines;

the gaseous pollutant is formaldehyde; and

the flow generated in step a) has a flow rate of between 0.1 μL/min and 100 μL/min.

The invention further provides a gaseous pollutant analysis device for implementing the method according to the invention, comprising a peristaltic pump, a container comprising a liquid solution comprising a selective derivatizing agent and having at least one inlet and one outlet, the outlet being connected to the peristaltic pump, a means for trapping and dissolving the gaseous pollutant in a liquid flow comprising the liquid solution, a means for reacting the gaseous pollutant with the selective derivatizing agent to form a derivative compound, connected to the inlet of the container and to the trapping means, and a sensor suitable for determining a concentration of derivative compound, connected to the peristaltic pump and to the trapping means.

According to embodiments:

the trapping means is sited in an emission cell sited on a surface of a material emitting the gaseous pollutant, and/or

the device is adapted so as to be a closed microfluidic circuit.

The invention also provides a gaseous pollutant analysis device for implementing the method according to the invention, comprising at least one inlet suitable for a solution comprising at least one liquid selective derivatizing agent, a peristaltic pump connected to the inlet, a means for trapping and dissolving the gaseous pollutant in a liquid flow comprising the derivatizing agent and sited at the outlet from the peristaltic pump, a means for reacting the gaseous pollutant with the derivatizing agent to form a derivative compound and sited at the outlet of the trapping means, a sensor suitable for determining a concentration of derivative compound, sited at the outlet of the reaction means, and at least one outlet suitable for evacuating the gaseous pollutant, the selective derivatizing agent and derivative compounds from the reaction between the gaseous pollutant and the selective derivatizing agent.

According to embodiments:

the device further comprises an inlet for a liquid and a system of solenoid valves which is sited between the inlets for the liquid and the selective derivatizing agent and the peristaltic pump in such a way that the outlet from the pump is a liquid flow comprising the derivative agent; and

the device further comprises an inlet and an outlet which are suitable for a gas comprising the gaseous pollutant.

According to embodiments relating to the devices of the invention:

the sensor comprises a fluorescence detector or a spectrometer or a colorimeter;

the trapping means comprises a microporous tube;

the trapping means is a microfluidic chip comprising a porous membrane and at least one inlet and one outlet which are suitable for a liquid;

the reaction means is a microfluidic chip; and

the sensor comprises a microfluidic chip.

Further features, details and advantages of the invention will become apparent from a reading of the description with reference to the appended figures, which are given by way of example and which represent respectively:

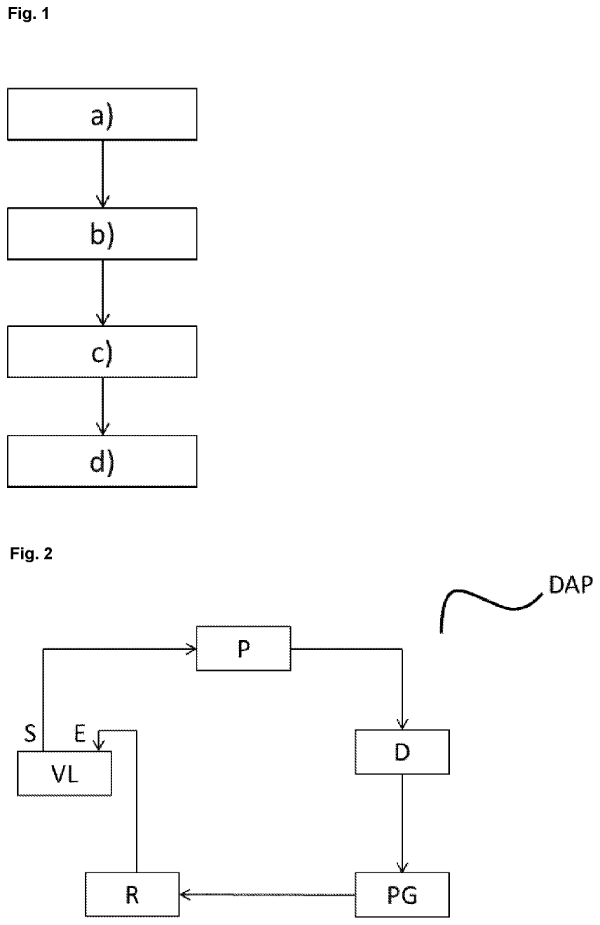

FIG. 1, a scheme of the steps in the method according to the invention;

FIG. 2, an analytical device according to a first embodiment of the invention;

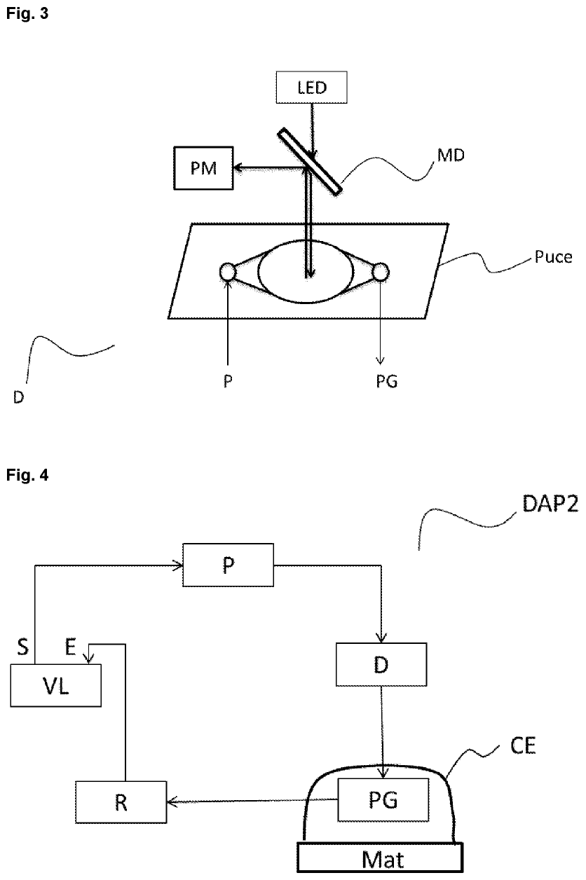

FIG. 3, an example of a means for determining the concentration of gaseous pollutant of the device according to the invention;

FIG. 4, an analytical device according to a second embodiment of the invention;

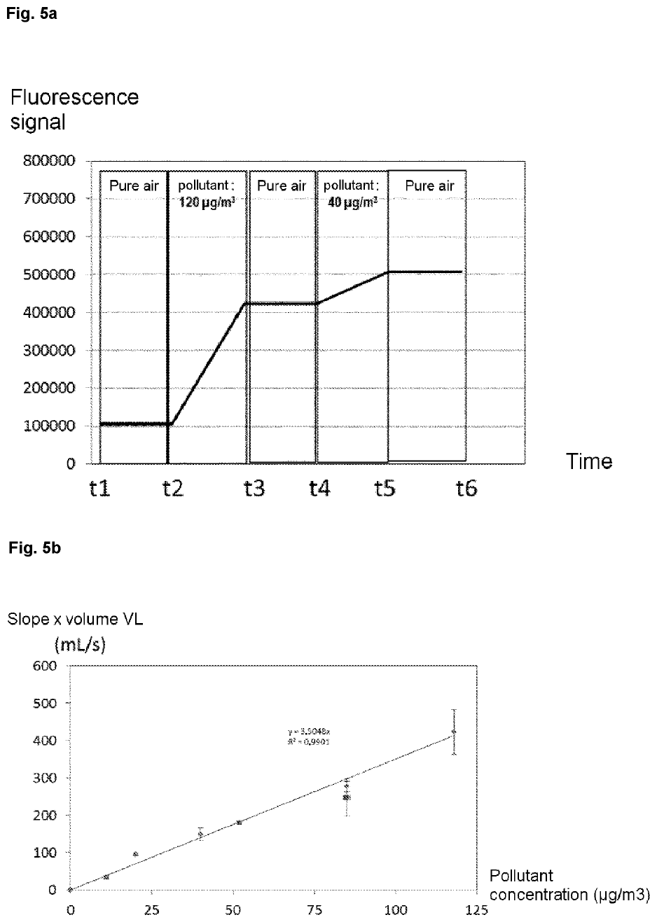

FIG. 5a and FIG. 5b, a measurement example carried out with the analytical device according to the first embodiment of the invention;

FIG. 6, an analytical device according to a third embodiment of the invention;

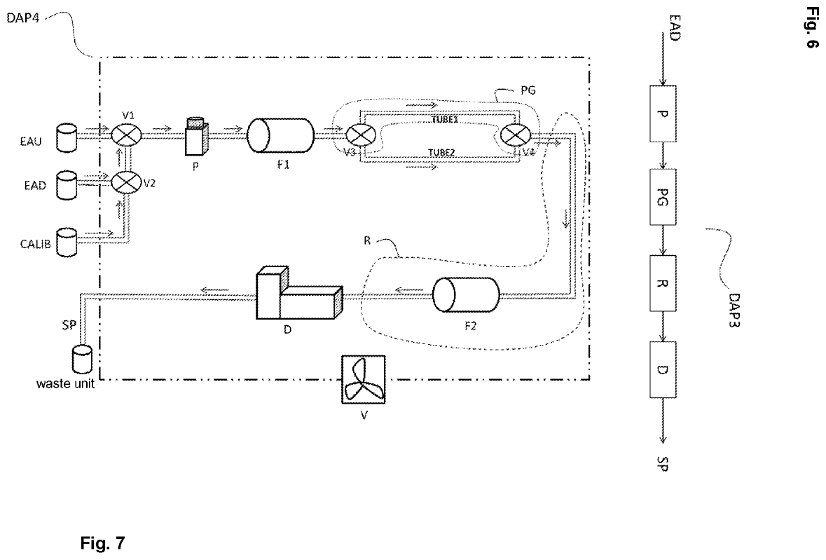

FIG. 7, an analytical device according to a fourth embodiment of the invention;

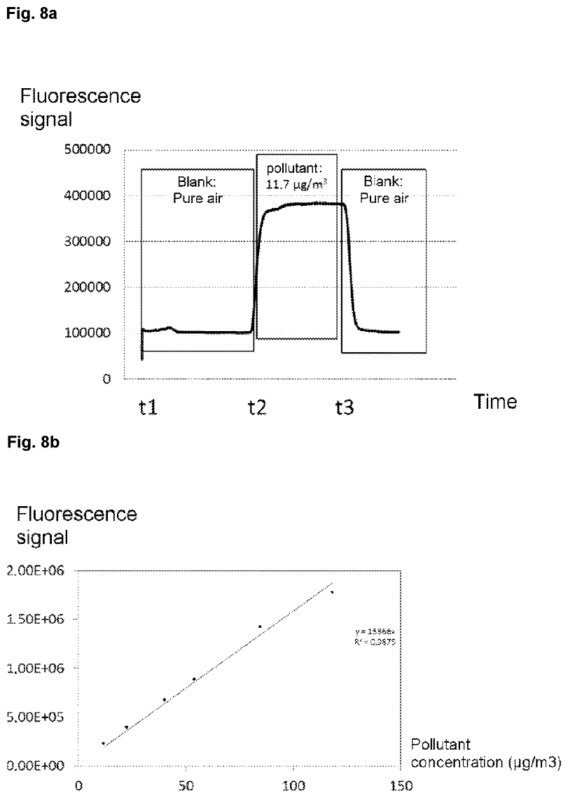

FIG. 8a and FIG. 8b, a measurement example carried out with the analytical device according to the third and fourth embodiments of the invention.

In the present invention, a selective derivatizing agent is a reagent which reacts with the gaseous pollutant for analysis to form a compound, called a derivative compound, which is readily detectable and quantifiable.

FIG. 1 represents a scheme of the steps of the method according to the invention. The method comprises four steps, from a) to d). It is implemented by means of a microfluidic circuit comprising a means for pumping a liquid and a means for trapping a gas. Examples of this type of microfluidic circuit are given with reference to FIGS. 2 to 6.

The first step a) involves generating a flow of a liquid, the flow comprising a selective derivatizing agent. The selective derivatizing agent may be included in the liquid or added to the liquid during the generation of the flow. The flow is generated by pumping the liquid by virtue of the liquid pumping means present in the microfluidic circuit.

The second step b) involves trapping the gaseous pollutant for analysis in the flow generated in step a) and dissolving it in the flow.

The pollutant is trapped, for example, by the trapping means of the microfluidic circuit. Trapping may in particular be carried out by passing the gaseous pollutant across a porous surface. The porous surface may be a porous membrane sited on a microfluidic cell in which the liquid flow runs. The porous surface may also be a microporous tube in which the liquid flow runs.

The third step, step c), involves reacting the gaseous pollutant in solution in the flow with an excess of the selective derivatizing agent, so as to form a derivative compound. The concentration of selective derivatizing agent is in large excess, and so is not determining. Accordingly the pollutant is converted quantitatively into derivative compound in this reaction step.

The following step (step d)) involves measuring the concentration of derivative compound for determining the concentration of gaseous pollutant.

According to one embodiment, this step d) is carried out by fluorescence spectroscopy or by colorimetry.

If the derivative compound is a fluorescent compound, fluorescence measurements may be made, by fluorescence spectroscopy, for example, for determining the concentration of derivative compound and thereby determining the concentration of gaseous pollutant.

It is likewise possible to determine the concentration of derivative compound by colorimetry measurements, so as to then subsequently determine that of gaseous pollutant, because the concentration of selective derivatizing agent is in large excess and is therefore not determining. Accordingly, the pollutant is converted quantitatively into derivative compound.

According to embodiments, the gaseous pollutant for analysis is a compound from the class of the aldehydes, especially formaldehyde, or a compound from the class of the chloramines. More generally, the gaseous pollutant for analysis is a gas which is readily soluble in a liquid phase, thus having a high Henry constant at more than 20 mol·L⁻¹·( 1/9·9e10⁻⁶)Pa⁻¹, or 20 M/atm, or having a rapid reaction in solution, despite a Henry constant of less than 20 M/atm.

The selective derivatizing compound is selected so as to react with the gaseous pollutant for analysis. Accordingly, if the gaseous pollutant is formaldehyde, the derivatizing compound might be, for example, Fluoral-P; or, if the gaseous pollutant is a chloramine, the derivatizing compound might be, for example, a mixture of iodine and starch.

The flow generated in step a) is preferably a slow flow: the slower the flow, the greater the quantity of gaseous pollutant dissolved per unit volume of derivatizing agent. If the flow is too fast, the dilution of the gaseous pollutant in the flow will be excessive. The flow is considered to be slow if the flow rate is between 0.1 μL/min and 100 μL/min, and more preferably if it is between 1 μL/min and 50 μL/min.

According to one embodiment, step c) comprises regulation of the temperature of the liquid flow so as to control the kinetics of reaction between the pollutant and the derivatizing agent and so to promote and/or to accelerate the reaction of the dissolved pollutant with the derivatizing agent.

According to another embodiment, step a) of the method further comprises a step of calibrating the microfluidic circuit, so as to determine a concentration of gaseous pollutant internal to the microfluidic circuit. This calibration step also enables calibration of the means for determining the concentration of gaseous pollutant—for example, the means for fluorescence or colorimetric measurement of the derivative compound.

FIG. 2 represents a gaseous pollutant analysis device DAP, according to a first embodiment of the invention, enabling implementation of the method of the invention.

The DAP device is a closed circuit which comprises a peristaltic pump P, a container VL suitable for comprising a liquid solution, a means PG for trapping the gaseous pollutant, a means R for reacting the gaseous pollutant with a selective derivatizing agent, and a sensor D suitable for determining the concentration of derivative compound, to then enable determination of the concentration of gaseous pollutant.

The container VL has at least one inlet E and one outlet S, and the liquid solution contained in the container VL comprises at least one selective derivatizing agent. The liquid solution may be a mixture of the derivatizing agent with another liquid, or just the derivatizing agent. The outlet S of the container VL is connected to the peristaltic pump P.

The trapping means PG is suitable for trapping the gaseous pollutant in a liquid flow comprising the liquid solution, the flow being generated by the peristaltic pump P. More particularly it generally comprises a microporous tube or a microporous membrane.

The reaction means R is connected to the inlet E of the container VL. The sensor D is connected to the peristaltic pump P and to the trapping means PG.

According to one embodiment, the trapping means PG is a microfluidic chip comprising at least one inlet and one outlet for the liquid flow and a porous membrane. The porous membrane is sited such that on one side of the membrane there is the liquid flow and on the other side there is the gaseous pollutant. The porous membrane and the liquid flow rate enable the gaseous pollutant to be trapped in the liquid flow circulating in the chip, and to be dissolved in this same flow. The inlet of the chip is in this case connected to the outlet of the container VL, while the outlet thereof is connected to the reaction means R.

According to one embodiment, the reaction means R is a microfluidic chip comprising an inlet, connected to the outlet of the trapping means PG, and an outlet, connected to the inlet E of the container VL. This chip may comprise a serpentine channel so that the dissolved gaseous pollutant and the derivatizing agent have time to react and to form a derivative compound.

According to one embodiment, the reaction means R is thermostatic so as to control the kinetics of reaction between the gaseous pollutant and the selective derivatizing agent. The DAP device may accordingly comprise an oven or a Peltier module, sited between the trapping means PG and the container VL, in which the reaction means R is sited, with the oven enabling the flow to be heated and the Peltier module enabling a constant flow temperature to be maintained.

According to one embodiment, the sensor D comprises a microfluidic chip comprising an inlet connected to the peristaltic pump P and an outlet connected to the trapping means PG.

FIG. 3 illustrates an example of a sensor D comprising a microfluidic chip. The microfluidic chip PUCE is connected, via its inlet, to the peristaltic pump P, and via its outlet to the trapping means PG. A light source LED is sited above the chip PUCE, and a dichroic mirror MD is sited between the chip PUCE and the light source LED. The mirror MD is sited so as to send the light rays from the source LED to the chip PUCE and to reflect the light rays, from the chip PUCE. The light source LED is, for example, a light-emitting diode. The sensor D further comprises a photomultiplier PM sited so as to receive to light rays reflected by the mirror DM and coming from the chip PUCE.

According to another embodiment, the source LED is sited in place of the photomultiplier PM of FIG. 3, and the photomultiplier PM is sited in place of the source LED in FIG. 3.

According to one embodiment, optical filters may be sited in front of the photomultiplier PM and in front of the source LED in order better to differentiate the light reflected from the source LED and the fluorescence signal originating from the chip PUCE.

This implementation is particularly suitable for fluorescence measurements and may be used when the derivative compound emits fluorescence.

When it has been calibrated, the photomultiplier PM enables determination of the concentration of derivative compound and hence the determination of the concentration of gaseous pollutant.

More generally, the photomultiplier PM may be replaced by a photodiode or by another photodetector.

More generally, the detection may be performed by fluorescence spectroscopy or absorption spectroscopy by a sensor D, thereby enabling a determination of the concentration of derivative compound and hence a determination of the concentration of gaseous pollutant.

According to another embodiment, the trapping means PG and reaction means R are each realized on a separate microfluidic chip, and the sensor D comprises another microfluidic chip separate from the trapping means PG and reaction means R. This allows a miniature device to be obtained, since a chip may measure 75×25×1.5 mm, for example.

According to another embodiment, the trapping means PG is a microporous tube containing a flow of the liquid comprising the selective derivatizing agent. The gaseous pollutant crosses the porous surface of the tube and is then trapped in the flow crossing the tube. The pollutant thus dissolves in the liquid flow.

FIG. 4 shows an analysis device DAP2 according to a second embodiment of the invention. The device DAP2 is also a closed circuit and comprises the same elements as those shown in FIG. 2, and also an emission cell CE, which houses the trapping means PG. The emission cell CE is sited on a surface of a material Mat emitting the gaseous pollutant for analysis.

The emission cell CE has a generally cylindrical shape, with its base applied to the material Mat being circular. This device DAP2 enables direct determination of the concentration of the gaseous pollutant emitted by the material Mat, and hence determination of the emissions from this material Mat. A calibration step outside the method enables the concentration determined at equilibrium in the emission cell CE to be connected to the degree of emission of gaseous pollutant by the material Mat, this emission level being normally used for classifying materials according to their emission of this gaseous pollutant. For example, for a material emitting formaldehyde, there are four classes according to the labelling in France: A+, A, B and C, with A+ corresponding to the class in which the emissions are the lowest.

Moreover, the smaller the height of the emission cell CE, the more rapid the diffusion of the gaseous pollutant toward the trapping means PG. As before, the trapping means, reaction means and means for determining the concentration of pollutant may be realized as or comprise microfluidic chips.

The devices DAP and DAP2 described operate in closed circuit. The liquid solution contained in the container VL becomes continually enriched with gaseous pollutant and therefore with derivative compound from the reaction between the derivatizing agent and the gaseous pollutant.

Where the derivative compound emits fluorescence, its concentration may be measured from the slope of the curve representing the increase in the fluorescence signal as a function of time.

Where absorbance measurements are performed on the derivative compound, the concentration of derivative compound will be determined from the slope of the curve representing the increase in absorbance as a function of time.

FIG. 5a and FIG. 5b present an example of a measurement of fluorescence emitted by the derivative compound. FIG. 5a represents the fluorescence signal of the derivative compound as a function of time, and FIG. 5b represents the product of the slope of the fluorescence signal and the volume of the container VL as a function of the concentration of gaseous pollutant.

Between t1 and t2, between t3 and t4 and between t5 and t6, the slope of the signal is zero, and therefore no additional derivative compound is formed in these time intervals. This means that the air around the trapping device does not contain the gaseous pollutant, and that the air is pure (analytical blank) in these same time intervals.

Between t2 and t3 and between t4 and t5, the slope of the fluorescence signal is increasing. This means that the solution placed in closed circuit is becoming enriched with gaseous pollutant, which reacts with the selective derivatizing agent to form the fluorescent derivative compound. There is therefore gaseous pollutant present around the trapping device. The greater the slope of the signal, the higher the concentration of gaseous pollutant in the air.

After having determined the concentration of derivative compound, then that of gaseous pollutant from the slope of the fluorescence signal, it is also possible, in FIG. 5b, to represent the product of the slope of the fluorescence signal and the volume of the container VL as a function of the concentration of gaseous pollutant. The slope is multiplied by the volume of the container so as to take account of the dilution effect associated with the volume of derivatizing agent in recirculation. This also allows a linear relation to be obtained between the fluorescence signal and the concentration of pollutant, hence enabling quantitative analysis of the concentration of pollutant.

The volume of the container VL containing the selective derivatizing agent may be adapted to the user's desired measuring time. Accordingly, if rapid and precise measurement of the concentration of pollutant is desired, a small volume is used for the container VL—nevertheless, this also carries a risk of rapid saturation of the device. This is because saturation is reached as soon as the detector is saturated by too high a concentration of the solution. If the detector permits, and also the photomultiplier PM, consideration may be given to reducing the gain so as to obtain a non-saturated signal, with this implying that a calibration has already been carried out for these new conditions. More simply, when saturation is close or has been reached, it is necessary to change the container VL and to put in its place a new container still comprising a liquid solution free of pollutant and comprising a selective derivatizing agent capable of reacting with the gaseous pollutant for analysis. It is preferable to change the container VL, rather than just empty it and then refill it with the liquid solution in order to avoid a step of cleaning the container VL. The volume of the microfluidic circuit of the device could be purged beforehand with the same solution in order to remove the derivative compound from the device.

The two devices DAP and DAP2, each combined in the method described with reference to FIG. 1, enable a very small amount of selective derivatizing agent to be used and remove the need to have an external waste unit to store the derivative compound, and the excess of derivatizing agent, and, where appropriate, the traces of unreacted gaseous pollutant.

The analytical device DAP is particularly suitable for carrying out measurements in a working situation. It is possible, for example, to use a volume of 6 mL of selective derivatizing agent in order to perform analyses for six days for an indoor environment with low-level pollution (formaldehyde concentration less than 15 μg/m³) or to use a volume of 6 mL of derivatizing agent to perform analyses for thirty hours for an environment with higher-level pollution (formaldehyde concentration about 120 μg/m³). It is likewise possible to use a volume of 24 mL of selective derivatizing agent per 24 h of exposure in a polluted environment (formaldehyde concentration greater than 500 μg/m³).

The analytical device DAP2 is particularly suitable for producers of materials, furniture or decorative coatings (paints and coatings, for example).

FIG. 6 presents an analytical device DAP3 according to a third embodiment of the invention.

The device DAP3 comprises at least one inlet EAD suitable for a liquid solution comprising at least one liquid selective derivatizing agent, a peristaltic pump P connected to the inlet EAD and a means PG for trapping the gaseous pollutant in the selective derivatizing agent, sited at the outlet from the pump P. Following the trapping means PG, a means R for reacting the gaseous pollutant with the selective derivatizing agent allows the two compounds to be reacted to form at least one derivative compound. A sensor D suitable for determining the concentration of derivative compound is connected following the reaction means R. The sensor D is connected to an outlet SP of the device DAP3 which allows the remaining derivative compound, dissolved gaseous pollutant and derivatizing agent to be evacuated. The device DAP3 operates in an open circuit, unlike the devices presented above.

The inlet EAD is suitable for receiving a liquid mixture of the selective derivatizing agent and other liquids, or for receiving solely the liquid selective derivatizing agent.

The peristaltic pump P enables pumping of the derivatizing agent and the generation of a liquid flow comprising this agent, so that the gaseous pollutant is subsequently trapped and then dissolved in this flow by virtue of the trapping means PG.

FIG. 7 presents a device DAP4 according to a fourth embodiment of the invention. This device DAP4 is of the same type as the device DAP3, as it also operates in an open circuit.

As well as the elements already present in the device DAP3, the device DAP4 comprises a system of solenoid valves V1, V2, V3 and V4, two other inlets EAU and CALIB, two ovens F1 and F2, two tubes TUBE1 and TUBE2, a waste unit at the outlet SP, and a fan. The oven F1 is optional. One of the tubes, TUBE1, forms the trapping means PG, while the second tube, TUBE2, is placed in parallel with the trapping means and can be used to calibrate the device DAP3.

The inlets EAU, EAD and CALIB are sited at the inlet of the peristaltic pump. A first solenoid valve V1 is sited between the inlet EAU and the pump, and a second solenoid valve V2 connects the inlets EAD and CALIB to the first solenoid valve V1. The inlets EAD and CALIB are each connected to one of the ports of the solenoid valve V2.

The inlet EAU allows a liquid, generally water, to be sent to the inlet of the peristaltic pump P. The inlet EAD is also connected to the pump P, and so it is then possible to generate a liquid flow, at the outlet from the pump P, that comprises the selective derivatizing agent or the liquid from the inlet EAU.

The inlet CALIB is generally used to calibrate the device. It is therefore suitable for receiving a mixture of the selective derivatizing agent and the gaseous pollutant for analysis.

At the outlet from the pump P, an oven F1 may optionally be present for the purpose of heating the liquid flow, which will have the effect of subsequently promoting the reaction between the derivatizing agent and the pollutant. In place of the oven F1, it is possible to have a temperature-regulating system, such as a Peltier system, to regulate the temperature of the liquid flow. This is especially useful when the gaseous pollutant is a chloramine, since the derivative compound resulting from chloramines undergoes decomposition at above 30° C. The system in that case will be configured to maintain a temperature of the flow at 20° C.

Following the oven F1 is the trapping means PG, formed by the two solenoid valves V3 and V4 and the tube TUBE1. The second tube, TUBE2, is placed in parallel with the trapping means. The first tube, TUBE1, is connected to one of the ports of the solenoid valve V3 and to one of the ports of the solenoid valve V4. The second tube, TUBE2, is connected to another port of the solenoid valve V3 and to another port of the solenoid valve V4. The oven F1 is connected to the trapping means PG by another port of the solenoid valve V3. Moreover, another port of the solenoid valve V4 connects the trapping means PG to the reaction means R.

The first tube, TUBE1, is a tube suitable for trapping the gaseous pollutant. It may therefore be a microporous tube, for trapping the gaseous pollutant in a liquid flow circulating in this tube TUBE1.

The second tube, TUBE2, is a tube which is unable to trap the gaseous pollutant. It is generally a nonporous tube carrying a flow only of the liquid from the inlet EAU, or only the selective derivatizing agent from the inlet EAD, or else the liquid mixture from the inlet CALIB. It is made, for example, of Teflon or of polyetheretherketone (PEEK). This second tube, TUBE2, will be selected more generally for calibration of the device DAP4 by a liquid solution sited at the inlet CALIB. This tube TUBE2 would also be selected for producing a blank, by selection either of the liquid from the inlet EAU or of the selective derivatizing agent from the inlet EAD.

The reaction means R is sited at the outlet of the trapping means PG. In this example it comprises an oven F2 which enables heating of the flow circulating in the reaction means R and comprising the gaseous pollutant dissolved in a liquid solution comprising at least the selective derivatizing agent. The oven F2 allows control of the reaction of the agent with the pollutant, and more particularly allows the reaction to be accelerated.

A system for regulating the temperature of the flow, such as a Peltier system, may also be present in place of the second oven F2.

The outlet from the reaction means R is connected to the sensor D suitable for determining the concentration of derivative compound, this sensor being itself connected to the outlet SP of the device DAP4. As for the preceding devices, this sensor D may be suitable for carrying out fluorescence or colorimetry measurements on the derivative compound formed by the reaction of the pollutant with the derivatizing agent.

The outlet SP from the device DAP4 generally forms a waste unit for evacuating the liquid solution comprising the derivative compound, the excess derivatizing agent, and optionally the dissolved, unreacted gaseous pollutant.

A fan V may advantageously be present and sited so as to ventilate the ambient air within the device DAP4, in order to promote the trapping of any gaseous pollutant by the trapping means PG.

The device DAP4 comprises two ovens F1 and F2, though it is also possible for only the oven F1 to be present or only the oven F2 to be present, or else for no oven to be present if the reaction does not require it.

Similarly, it is also possible for two systems for regulating the temperature of the flow to be present, in the place of F1 and F2, or for only one system for regulating the temperature of the flow to be present, in place of F1 or of F2.

According to another embodiment, the device DAP4 comprises an inlet suitable for a gas, especially for the gaseous pollutant, and an outlet suitable for a gas. This inlet and this outlet are used in order to calibrate the device DAP4 and, more particularly, to inject a mixture of known concentration at very low flow rates around the tubes TUBE1 and TUBE2. This type of calibration enables a determination of the trapping yield and hence produces measurements which are more precise, in order for the concentration of gaseous pollutant in the ambient air in the device DAP4 to be determined with the greatest possible precision.

The two devices DAP3 and DAP4 are particularly suitable for specialists in the metrology of airborne pollutants, owing to their measurement precision.

FIG. 8a and FIG. 8b present a measurement example carried out with the device DAP3 and DAP4 of the third and fourth embodiments.

A fluorescence signal emitted by the derivative compound formed by the reaction of the gaseous pollutant with the selective derivatizing agent, and detected by the determination means D, is represented as a function of time. FIG. 8a represents this fluorescence signal as a function of time, while FIG. 8b represents the same fluorescence signal as a function of the concentration of pollutant in the ambient air.

Between t1 and t2, a blank is performed with pure air, using the gas mode (utilization of inlets and outlets dedicated to a gas). The solenoid valves V3 and V4 are therefore configured so that the liquid flow circulates only in the first TUBE1, which is suitable for trapping the gaseous pollutant. Since pure air is injected, the device DAP4 is unable to become enriched in gaseous pollutant. The fluorescence signal of the derivative compound is therefore constant.

Subsequently, between t2 and t3, the device DAP4 goes into measurement mode. The solenoid valves V3 and V4 are still configured so that the liquid flow circulates in the first tube TUBE1 suitable for trapping the gaseous pollutant. During this interval of time, if gaseous pollutant is indeed present, the fluorescence signal increases until a plateau is reached (as in the case shown in this figure). Given the height of this signal relative to the blank carried out beforehand, it is possible to determine the concentration of gaseous pollutant on the basis of a calibration performed earlier.

Between t3 and t4, operation switches back to blank mode, with pure air. Accordingly, the device cannot be supplied with any gaseous pollutant surrounding the device, and the fluorescence signal decreases, since the derivative compound obtained before is removed via the outlet SP, until a constant level is reached.

It is also possible to run another, virtually identical, sequence, but using the second tube TUBE2 for performing the blank between t1 and t2 and between t3 and t4 and performing the measurements between t2 and t3 as explained in the preceding paragraph.

After the concentration of gaseous pollutant has been determined on the basis of the fluorescence signal, it is possible to represent, in FIG. 8b, the fluorescence signal as a function of the concentration of gaseous pollutant in the ambient air around the trapping means PG. It is observed that these two quantities are linked to one another by a linear relationship, hence allowing quantified analyses to be made.

1. A method for analyzing a gaseous pollutant by means of a microfluidic circuit comprising a means for pumping a liquid and a means for trapping a gas, comprising the following steps:
a) generating a flow of a liquid, the liquid comprising a selective derivatizing agent;
b) trapping and dissolving the gaseous pollutant in the flow;
c) reacting the pollutant with the selective derivatizing agent to form a liquid derivative compound;
d) measuring the concentration of liquid derivative compound and determining the concentration of gaseous pollutant.

2. The method for analyzing a gaseous pollutant as claimed in claim 1, wherein step c) comprises temperature-regulating the liquid flow.

3. The method for analyzing a gaseous pollutant as claimed in claim 1, wherein step d) is carried out by fluorescence spectroscopy or by colorimetry.

4. The method for analyzing a gaseous pollutant as claimed in claim 1, wherein the gaseous pollutant is selected from a compound from the class of the aldehydes or a compound from the class of the chloramines.

5. The method for analyzing a gaseous pollutant as claimed in claim 1, wherein the gaseous pollutant is formaldehyde.

6. The method for analyzing a gaseous pollutant as claimed in claim 1, wherein the flow generated in step a) has a flow rate of between 0.1 μL/min and 100 μL/min.

7. A gaseous pollutant analysis device (DAP) for implementing the method as claimed in claim 1, comprising:
a peristaltic pump (P);
a container (VL) comprising a liquid solution comprising a selective derivatizing agent, and having at least one inlet (E) and one outlet (S), the outlet being connected to the peristaltic pump;
a means (PG) for trapping and dissolving the gaseous pollutant in a liquid flow comprising the liquid solution;
a means (R) for reacting the gaseous pollutant with the selective derivatizing agent to form a derivative compound, connected to the inlet (E) of the container (VL) and to the trapping means (PG); and
a sensor (D) suitable for determining a concentration of derivative compound, connected to the peristaltic pump (P) and to the trapping means (PG).

8. The gaseous pollutant analysis device (DAP2) as claimed in claim 7, wherein the trapping means (PG) is sited in an emission cell (CE) sited on a surface of a material (Mat) emitting the gaseous pollutant.

9. The gaseous pollutant analysis device (DAP, DAP2) as claimed in claim 7, adapted so as to be a closed microfluidic circuit.

10. A gaseous pollutant analysis device (DAP3) for implementing the method as claimed in claim 1, comprising:
at least one inlet (EAD) suitable for a solution comprising at least one liquid selective derivatizing agent;
a peristaltic pump (P) connected to the inlet;
a means (PG) for trapping and dissolving the gaseous pollutant in a liquid flow comprising the derivatizing agent, sited at the outlet from the peristaltic pump;
a means (R) for reacting the gaseous pollutant with the derivatizing agent to form a derivative compound, sited at the outlet of the trapping means;
a sensor (D) suitable for determining a concentration of derivative compound, sited at the outlet of the reaction means; and
at least one outlet (SP) suitable for evacuating the gaseous pollutant, the selective derivatizing agent and derivative compounds from the reaction between the gaseous pollutant and the selective derivatizing agent.

11. The gaseous pollutant analysis device (DAP4) as claimed in claim 10, further comprising an inlet for a liquid (EAU) and a system of solenoid valves (V1, V2) which is sited between the inlets for the liquid and the selective derivatizing agent and the peristaltic pump in such a way that the outflow from the pump is a liquid flow comprising the derivative agent.

12. The gaseous pollutant analysis device as claimed in claim 10, further comprising an inlet and an outlet which are suitable for a gas comprising the gaseous pollutant.

13. The gaseous pollutant analysis device (DAP, DAP2, DAP3, DAP4) as claimed in claim 7, wherein the sensor (D) comprises a fluorescence detector or a spectrometer or a colorimeter.

14. The gaseous pollutant analysis device (DAP, DAP2, DAP3, DAP4) as claimed in claim 7, wherein the trapping means (PG) comprises a microporous tube (TUBE1).

15. The gaseous pollutant analysis device (DAP, DAP2, DAP3) as claimed in claim 7, wherein the trapping means is a microfluidic chip comprising a porous membrane and at least one inlet and one outlet which are suitable for a liquid.

16. The gaseous pollutant analysis device (DAP, DAP2, DAP3) as claimed in claim 7, wherein the reaction means (R) is a microfluidic chip.

17. The gaseous pollutant analysis device (DAP, DAP2, DAP3) as claimed in claim 7, wherein the sensor (D) comprises a microfluidic chip (PUCE).
